SEQUENCE LISTING (13 sequences)

SEQ ID NO: 1 - DNA, 68 bp, Artificial Sequence
  Description of Combined DNA/RNA Molecule DNA/2′OMeRNA derived from the murine gene encoding the cGMP- phosphodiesterase beta-subunit
ccttccaacctacgtagcagaaagtttttacuuucugcuacgtagguuggaagggcgcgttttcgcgc

SEQ ID NO: 2 - DNA, 25 bp, Artificial Sequence
  Description of Artificial Sequence DNA derived from the murine or human gene encoding the transcription factor HIF1alpha
ccatgtgaccattaggaaatgagag

SEQ ID NO: 3 - DNA, 24 bp, Artificial Sequence
  Description of Artificial SequenceDNA derived from the human gene encoding the opsin
gctttctttgccaagagcgccgca

SEQ ID NO: 4 - DNA, 22 bp, Artificial Sequence
  Description of Artificial Sequence DNA derived from the human gene encoding the RP1
aagaaaaaatctagacaagcaa

SEQ ID NO: 5 - DNA, 25 bp, Artificial Sequence
  Description of Artificial Sequence DNA derived from the murine gene encoding the opsin
gctttctttgctgagagctcttcca

SEQ ID NO: 6 - DNA, 22 bp, Artificial Sequence
  Description of Artificial Sequence DNA derived from the murine gene encoding the RP1
aagacttctgagtaacaatcaa

SEQ ID NO: 7 - DNA, 25 bp, Artificial Sequence
  Description of Artificial Sequence DNA derived from the murine or human gene encoding the transcription factor HIF1alpha
ccatgtgaccatgaggaaatgagag

SEQ ID NO: 8 - DNA, 24 bp, Artificial Sequence
  Description of Artificial Sequence DNA derived from the human gene encoding the opsin
gctttctttgccgagagcgccgca

SEQ ID NO: 9 - DNA, 15 bp, Artificial Sequence
  Description of Artificial Sequence DNA derived from the human gene encoding the RP1
aagaaaaaatcttga

SEQ ID NO: 10 - DNA, 25 bp, Artificial Sequence
  Description of Artificial Sequence DNA derived from the human gene encoding the RP1
gctttctttgctaagagctcttcca

SEQ ID NO: 11 - DNA, 68 bp, Artificial Sequence
  Description of Combined DNA/RNA Molecule DNA/2′OMeRNA derived from the murine gene encoding the cGMP- phosphodiesterase beta-subunit
ctaccaaatccatgggatttccatcagttauuucuguccatcagguaggagugggctcgcgtgcgttc

SEQ ID NO: 12 - DNA, 21 bp, Artificial Sequence
  Description of Artificial Sequence oligonucleotide sens (primer) derived from the murine cDNA encoding the cGMP-phosphodiesterase beta-subunit
ggccgggaaattgtcttctac

SEQ ID NO: 13 - DNA, 21 bp, Artificial Sequence
  Description of Artificial Sequence oligonucleotide reverse(primer) derived from the murine cDNA encoding the cGMP-phosphodiesterase beta-subunit
ccccaggaactgtgtcagaga

(19) United States
(12) Patent Application Publication
(10) Pub. No.: US 2003/0045830 A1
(43) Pub. Date: Mar. 6, 2003

(54) Gene therapy with chimeric oligonucleotides delivered by a method comprising a step of iontophoresis

(76) Inventors: Therese de Bizemont, Creteil (FR); Florian Sennlaub, Paris (FR); Francine BEHAR-COHEN, Paris (FR); Yves Courtois, Boulogne (FR)

Correspondence Address: BURNS DOANE SWECKER & MATHIS L L P, POST OFFICE BOX 1404, ALEXANDRIA, VA, 22313-1404, US

(21) Appl. No.: 09/836,439

(22) Filed: Apr. 17, 2001

(51) Int. Cl. A61K 48/00; C12N 15/87

(52) U.S. Cl. 604/20; 514/44; 435/461

(57) ABSTRACT

The present invention provides a method for enhancing the in vivo delivery of chimeric oligonucleotides, containing for example DNA/2′OMeRNA, into cells of a plant, an animal or a human, comprising a step of applying topically to or injecting into a tissue, or tissue adjacent to a tissue, containing said cells, a composition comprising said chimeric oligonucleotide, followed by, preceded by, or simultaneously to a step of transferring said chimeric oligonucleotide into said cells by iontophoresis, and relates to a gene therapy method comprising the iontophorically transfer of a chimeric oligonucleotide DNA/2′OMeRNA. The present invention is also directed to particular chimeric oligonucleotides DNA/2′OMeRNA capable of inducing or inhibiting the expression of a specific gene involved in eye function by inducing or reverting a mutation in that specific gene, and their use as therapeutic composition for preventing or treating ocular diseases.

Gene therapy with chimeric oligonucleotides delivered by a method comprising a step of iontophoresis

### FIELD OF THE INVENTION

The present invention provides a method for enhancing the in vivo delivery of chimeric oligonucleotides, containing for example DNA/2′OMeRNA, into cells of a plant, an animal or a human, comprising a step of applying topically to or injecting into a tissue, or tissue adjacent to a tissue, containing said cells, a composition comprising said chimeric oligonucleotide, followed by, preceded by, or simultaneously to a step of transferring said chimeric oligonucleotide into said cells by iontophoresis.

In particular, the invention relates to a gene therapy method of treating human eye affections, notably inherited retinopathics, comprising the iontophorically transfer of a chimeric oligonucleotide DNA/2′OMeRNA into eye tissue cells.

The present invention is also directed to particular chimeric oligonucleotides DNA/2′OMeRNA capable of inducing or inhibiting the expression of a specific gene involved in eye function by inducing or reverting a mutation in that specific gene, and their use as therapeutic composition for preventing or treating ocular diseases due in particular to the presence of a mutation, such as mutation present in the gene encoding the cGMP-phosphodiesterase β-subunit, said mutation leading to the murine retinitis pigmentosa disease, or mutation present in the RP1 or opsin gene, implicated in vision,

### BACKGROUND OF THE INVENTION

Gene therapy is the introduction of nucleic acid into a cell or tissue either in vivo or ex vivo. In some instances, the nucleic acid is intended to replace (or act in place of) or to correct a functionally deficient endogenous gene, to confer on the host the ability to produce a therapeutic polypeptide, to cause repression of an undesirable gene product, or to stimulate an immune response.

Among the process allowing the correction of a functionally deficient endogenous gene in eukaryotic cell, or to inactivate an undesirable gene, chimeraplasty has been the object of recent interest and has been cited as a potential process for the treatment of human disease and the development of useful, genetically engineered plant and animal strains (see for example the patent document U.S. Pat. No. 6,010,907 issued Jan. 4, 2000).

Chimeraplasty which has been defined for example in the patent document U.S. Pat. No. 5,565,350 issued Oct. 15, 1996, concerns the introduction of directed alterations in a specific site of the DNA of a target cell by introducing oligonucleotides, which are supposed to process by the cell's homologous recombination and repair systems so that the sequence of the target DNA is converted to that of the DNA pan of tie oligonucleotide. In order to effect a genetic change there are within the region of homology one or more non-corresponding base pairs (“heterologous” or “mutator” base pairs). It is thought that the cellular processes such as homologous recombination cause the mutator nucleotides to be inserted into the targeted genomic site. So, these oligonucleotides (hereinafter “chimeric oligonucleotides”) can be used to alter specifically a gene of interest by introducing into the gene the heterologous base pairs. The heterologous base pairs can be base pairs that changes the ones of the gene of interest, or base pairs in addition to those present in the gene of interest (an insertion), or the heterologous base pairs can induce the absence of base-pairs found in the gene of interest (a deletion).

The chimeric oligonucleotides generally contain ribo-type, e.g., 2′-O-methyl-ribonucleotides, and deoxyribo-type nucleotides that were designed to hybridize to each other. Among these chimeric oligonucleotides, the chimeric oligonucleotides designed with two blocks of 2′O-methyl RNA residues flanking a stretch of DNA, poly(T) hairpin loops and a G-C clamp for chemical and thermal stability as well as resistance to helicases and RNA- and DNA-nucleases and wherein part of the RNA/DNA sequence is complementary to that of the target gene, except that it contains at least single mismatched nucleotide in the DNA stretch when aligned with the homologous genomic DNA sequence can be particularly cited.

Different documents relating to the correction of a functionally deficient (endogenous) gene in eukaryotic cells using chimeraplasty have been published such as:

for correcting a mutation in the gene encoding liver/bone/kidney type alkaline phosphatase (Yoon, K., et al., 1996, Proc. Natl. Acad. Sci. 93, 2071);

for correcting a mutation in the human beta-globin gene that causes Sickle Cell Disease (Cole-Strauss, A., et al., 1996, Science 273, 1386); and

for the treatment of genetic diseases of hematopoictic cells, e.g., Sickle Cell Disease, Thalassemia and Gaucher Disease (U.S. Pat. No. 5,760,012 issued Jun. 2, 1998; PCT application No. WO 97/41141 filed Nov. 6, 1997; and U.S. Pat. No. 5,888,983 issued Mar. 30, 1999).

Generally, the strategy termed chimeraplasty has been used to correct or to create single-nucleotide mutations in genomic DNA. In this case, the part of the RNA/DNA sequence complementary to that of the target gene contains a single mismatched nucleotide in the DNA stretch when aligned with the homologous genomic DNA sequence. This unpaired nucleotide is apparently recognized by endogenous repair systems, thus changing the DNA sequence of the targeted mutated (wild type) gene back into its correct (in a mutated) version, These chimeric oligonucleotides have thus already been shown to be effective in introducing single-nucleotide conversion into several genes involved in pathological processes such as follows in table 1:

TABLE 1

| Disease | Targeted gene |
|---|---|
| Sickle cell disease | Hemoglobin βs |
| Hemophilia B | Factor IX |
| Crigler-Najjar Syndrome (type 1) | UDP-glucuronosyltransferase |
| Albinism | Tyrosinase |
| Duchenne muscular dystrophy | Dystrophine |

Nevertheless, in these above disclosed methods using chimeraplasty for gene therapy, the introduction of specific alterations in the genome of cells has been generally carried on by removing the cells containing the deficient gene from the subject, introducing the chimeric oligonucleotide (optionally with a step of culturing the removed cells) and reintroducing the cells into the subject.

Methods are known for introducing drug, such as nucleic acid, into target cells or tissues such as by topically applying to or injection into tissue the use of techniques such as electroporation, iontophoresis, the provision of nucleic acid in liposomes or other chemical carrier or the use of a viral or not viral vector.

While a lot of knowledge has been accumulated over the years, however, there are many problems that are often associated with the in vivo introduction of nucleic acid into eukaryotic cells by conventional methods, Typically only a small percentage of target cells desired to be transfected with heterologous nucleic acid actually express the gene of interest at satisfying levels, notably the protein of interest. In addition, some therapeutic compositions, such as those that include synthetic oligonucleotides, are very expensive, toxic and degradable, and, consequently, require very localized application and efficient internalization into the target cells. All those therapeutics require frequent administrations, contrary to chimeroplasty which is designed to induce a permanent gene modification.

Among the methods allowing to enhance the in vivo transfer of nucleic acid into target cells, electroporation can be particularly cited Electroporation means increased permeability, of a cell membrane and/or at least a portion of cells of A targeted tissue, to a chemical agent such as nucleic acids, wherein the increased permeability is caused by application of high pulse voltage across the cell or at least a portion of the tissue. The increased permeability allows transport, or migration, of chemical agents through the tissue or across cell membranes into cells if the tissue or the cells are in the presence of a suitable chemical agent. So, electroporation has been recently used to deliver nucleic acids to tissue.

Examples of nucleic acids delivery using electroporation methods are disclosed for example in following patent documents

U.S. Pat. No. 5,749,847 issued May 12, 1998, which disclosed the delivery of antisense oligonucleotide into epidermis after topically applying by electroporation for the treatment of melanomas; and

U.S. Pat. No. 6,110,161 issued Aug. 29, 2000, which disclosed the delivery of DNA after injection into epidermis by electroporation for genetic immunization.

Electroporation is typically carried out by applying high voltage pulses between a pair of electrodes which are applied to the tissue surface. The voltage must be applied in proportional to the distance between the electrodes. When the space between the electrodes is too great, the generated electric field penetrates deep into the tissue where it causes unpleasant nerve and muscle reaction.

Iontophoresis is a technique which was proposed in 1747 by Verrati and consists in the administration, in particular of medicaments, into the body through the tissues using an electric field involving a small voltage. An electrode is arranged at the site to be treated while a second electrode, intended to close the electric circuit, is placed at another site on the body. The electric field facilitates the migration of the active products, and/or increase cellular permeability to the products which are preferably ionized. This technique is commonly used for treating skin or rhumatologic diseases, and for this purpose there are a variety of devices which have been disclosed (part of them are available on the market) (see for example the patent documents U.S. Pat. No. 4,141,359 issued Feb. 27, 1979; U.S. Pat. No. 4,250,878 issued Jan. 17, 1981; U.S. Pat. No. 4,301,794 issued Nov. 24, 1981; U.S. Pat. No. 4,747,819 issued Apr. 31, 1988; U.S. Pat. No. 4,752,285 issued Jun. 21, 1988 ; U.S. Pat. No. 4,915,685 issued Apr. 10, 1990; U.S. Pat. No. 4,979,938 issued Dec. 25, 1990; U.S. Pat. No. 5,252,022 issued Oct. 5, 1993; U.S. Pat. No. 5,374,245 issued Dec. 20, 1994; U.S. Pat. No. 5,498,235 issued Mar. 12, 1996; U.S. Pat. No. 5,730,716 issued Mar. 24, 1998; U.S. Pat. No. 6,001,088 issued Dec. 14, 1999; U.S. Pat. No. 6,018,679 issued Jan. 25, 2000; I1.U.S. Pat. No. 6,139,537 issued Oct. 31, 2000; U.S. Pat. No. 6,148,231 issued Nov. 14, 2000; U.S. Pat. No. 6,154,671 issued Nov. 28, 2000, and U.S. Pat. No. 6,167,302 issued Dec. 26, 2000).

Another published document related to the ex-vivo oligonucleotides transfer into eye rabbit for the induction of genes can be also cited (Asahara et al., Nippon Ganga Gakkai Zasshi, 103 (3), 178-185, 1999).

It is known that iontophoresis wherein low voltage is applied between widely spaced electrodes can transport charged molecules through existing pathways and/or creating pathways. However, it is also known that the volumes of molecules transported is very small, and insufficient for in vivo applications in specific tissues. In order to overcome this remaining problem, a method has been particularly disclosed comprising the simultaneous use of both electroporation and iontophoresis for acid nucleic delivery in the patent document U.S. Pat. No. 6,009,345 issued Dec. 28, 1999.

From the foregoing, it will be appreciated that it would be an advancement in the art to provide a simple and efficient method for enhancing the in vivo delivery of nucleic acid, such as chimeric oligonucleotide, into target cells, particularly for ocular gene therapy.

Such a method is the object of the present invention which is disclosed herein.

Indeed, the inventors have shown for the first time that iontophoresis can be used only to efficiently enhance chimeric oligonucleotides penetration into target cells in vivo, notably after or during, or prior to intra-tissue injection of said chimeric oligonucleotides, thus allowing a more simply, efficiently and widely use of the chimeraplasty for gene therapy in vivo.

### SUMMARY OF THE INVENTION

The present invention relates to a new method for in vivo delivering a nucleic acid, preferably a chimeric oligonucleotide DNA/2′OMeRNA type, into target cells of an organism, preferably a mammal organism, including the step of topically applying to or injecting into that organism tissue, or tissue adjacent to a tissue containing said target cells, a composition comprising said desired nucleic acid followed by, or preceded by, or during the step of transferring said nucleic acid into said cells by iontophoresis.

In a preferred embodiment, the composition comprising said desired nucleic acid is injected into the tissue containing the target cells or into a joint space or tissue adjacent to said target cells.

In a preferred embodiment, said target cells are cells of an eye tissue, skeletal muscle, subcutaneous cells, or epidermal cells.

In another preferred embodiment, the method for delivering in vivo a nucleic acid into target cells according to the present invention is used to treat or to prevent an ocular disease, such as inherited retinopathies, due to the presence of at least a mutation in a gene of that target cells, mutated gene whose expression is responsible for said ocular disease. In this method, said nucleic acid is complementary to a genomic DNA fragment sequence of the target mutated gene of said cells with the exception of the mutation which is desired to be reverted in said target mutated gene.

In another preferred embodiment, the method for delivering in vivo a nucleic acid into target cells according to the present invention is used to voluntary induce a mutation in a gene of that target cells of an animal, mutated gene whose expression is responsible for an ocular disease, in order to obtain an animal, or an animal or human tissu or organism which can serve as a model for studying said ocular disease or for screening compounds capable of treating that ocular disease.

The present invention is also directed to a composition, particularly a pharmaceutical composition containing chimeric oligonucleotide DNA/2′OMeRNA type having or comprising a sequence selecting from the group of the sequences SEQ ID No. 1 to 6, wherein at least part of that DNA/RNA sequence is complementary to a genomic DNA fragment sequence of a target gene, preferably mutated, with the exception of the mutation, nucleotide or sequence fragment which is desired to be reverted, modified, added or inserted in said target gene, said target gene being selected from the group consisting of:

the gene encoding the cGMP-phosphodiesterase β-subunit, wherein the non-sens C→A mutation in position nt 347 of the cDNA of part of this gene leads to the murine retinitis pigmentosa disease,

the RP1 gene, wherein a missense or a nonsense mutation in that rhodopsin gene produces a non-functional protein, such as opsin protein; and

the gene encoding the transcription factor HIF1α which governs the expression of several genes involved in inflammation and neovascularization, and wherein induced nonsense mutation leads to a protein which will not be able to promote hypoxia induced neovascularization.

The present invention is finally directed to method to treat disease associated to the presence of mutation or disease which can be treated by a mutation induced among these above-cited target genes comprising the in vivo administration of the in the chimeric oligonucleotide DNA/2′OMeRNA type according to the present invention.

### BRIEF DESCRIPTION OF THE FIGURES

FIGS. 1A to 1C: Histochemistry of biotinylated chimeroplast and hemalun staining

FIG. 1A: Control retina.

FIG. 1B: Retina after injection into the vitreous of the biotinylated chimeroplast, No staining is observed in the retina or in the RPE showing that no chimeroplast has penetrated into the retina.

FIG. 1C: Retina after injection into the vitreous of the chimeroplast, followed by the iontophoresis of saline. There is a clear brown DAB staining in the retinal layers, in the RPE and in the choroid, showing that the penetration of the chimeroplast has been enhanced by the application of the current.

FIG. 2: Restriction fragment length analysis of β-cGMP phosphodiesterase cDNA

RT-PCR were performed with rd β-PDE mRNA specific primers on extracted retinae at postnatal day 27 (except for lanes 4-7 analyzed at postnatal day 10). The rd nonsense point mutation in codon 347 creates a DdeI restriction site and removes a BsaAI site from the wild-type sequence. Digesting the 359 bp β-PDE cDNA with BsaAI or DdeI yields two diagnostic fragments of 120 bp and 239 bp. This method allows the differentiation of the mutated sequence rd/rd (DdeT sensitive) from the wild-type one +/+ (BsaAI sensitive) at the mRNA level.

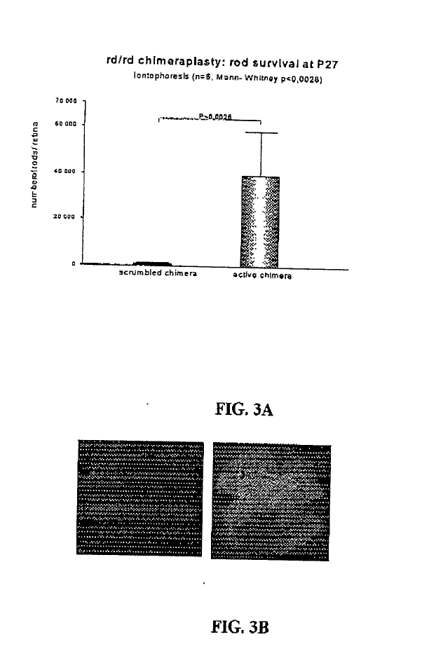

The gel in FIG. 2 represents the restriction fragment length analysis by electrophoresis separation:

lanes 1-3: for the wild-type cCDA sequence (+/+) without treatment; and

lanes 4-18for the mutated sequence (rd/rd) without treatment (lanes 4-6), with water injection treatment (lanes 7-9), with chimeroplast injection without iontophoresis transfer (lanes 10-12), with chimeroplast injection with iontophoresis transfer (lanes 13-15), with control chimeroplast injection with iontophoresis transfer (lanes 16-18).

FIGS. 3A and 3B: Rod survival by immunostaining

FIG. 3A: The amount of rod-photoreceptors was counted on flat-mounted retina of chimeraplast treated animals (“active chimera”) and control (“scrumbled chimera”) at postnatal day 27 (P27). Results were expressed as mean ± standard error of the mean (SEM).

FIG. 3B: Opsin-immunohistochemistry has been performed on whole-mounted retina. Scanned photograph by fluorescence microscopy of flat-mounted retina of chimeraplast treated animals (“active chimera”, right picture) and control (“scrumbled chimera”, left picture).

### DETAILED DESCRIPTION OF THE INVENTION

The present invention is directed to a method for delivering in vivo a nucleic acid, preferably a chimeric oligonucleotide, into target cells of an animal or human tissue, comprising the steps of:

a) topically applying to or injecting into the patient tissue, or a patient tissue adjacent to the patient tissue containing said target cells, a composition comprising said nucleic acid; and

b) transferring said nucleic acid into said target cells by iontophoresis.

In the method of the present invention, step b) can be carried out prior to, during or after the step a).

In the present specification the term “nucleic acid” is understood to mean an isolated natural, or a synthetic, a DNA and/or RNA fragment comprising natural and/or non natural nucleotides, designating a precise succession of at least 10, 15, 20, 25, 30, 35, 40, 45, 50, 75, 100 nucleotides, optionally modified. Said nucleic acid can be under the form of one strand, two strands or more, linear or circular and eventually closed.

In the present specification, the terms “chimeric oligonucleotide” is understood to mean a nucleic acid compounds capable of introducing specific genetic alterations into living eukaryotic cells as defined in the U.S. Pat. No. 5,565,350 issued Oct. 15, 1996 (incorporated herein by reference). Said “chimeric oligonucleotide” is defined as a polynucleotide having both ribonucleotides, modified or not and deoxyribonucleotides in a first strand and solely deoxyribonucleotides in a second strand wherein the strands have a Watson-Crick complementarity and are linked by oligonucleotides so that the polynucleotide has at most a single 3′ and a single 5′ end, and wherein these ends can be ligated so that the polynucleotide is a single continuous circular polymer.

With respect to the U.S. Pat. No. 5,565,350, these “chimeric oligonucleotides” used to induce specific alterations in targeted genes can be also defined by claim one of that patent as following.

Mixed ribo-deoxyribonucleic acid having at most one 3′ end and one 5′ end, which nucleic acid further comprises:

a) at least one region of contiguous unpaired bases disposed so that the unpaired region separates the nucleic acid into a first strand and a second strand;

b) connected by said region of contiguous unpaired bases a region of Watson-Crick nucleic acid of at least 15 base pairs in length, in which bases of the first strand correspond to bases of the second strand, and in which;

c) the first strand comprises a region of at least three contiguous nucleotides comprised of a 2′-O or 2′-OMe ribose, which form a hybrid-duplex within the region of Watson-Crick nucleic acid.

So, the chimeric oligonucleotides generally contain ribo-type, e.g., 2′-O-methyl-ribonucleotides, and deoxyribo-type nucleotides that were complementary according to Watson-Crick rules. Among these chimeric oligonucleotides, the chimeric oligonucleotides designed with two blocks of, preferably 10, 2′O-methyl RNA residues flanking a stretch of, preferably pentameric, DNA, poly(T) hairpin loops and a G-C clamp for chemical and thermal stability as well as resistance to helicases and RNA- and DNA-nucleases and wherein part of the RNA/DNA sequence is complementary to that of the target gene, except that it contains at least single mismatched nucleotide in the DNA stretch when aligned with the homologous genomic DNA sequence, are preferred.

Also are preferred, the “chimeric oligonucleotides” disclosed in the above cited documents relating to the correction of a functionally deficient gene, or to the creation of a deficient gene, in an eukaryotic cell using chimeraplasty which are incorporated herein by reference (Yoon, K., et al., 1996, Proc. Natl. Acad. Sci. 93, 2071; Cole-Strauss, A., et al., 1996, Science 273, 1386; U.S. Pat. No. 5,760,012 issued Jun. 2, 1998; PCT application No. WO 97/41141 filed Nov. 6, 1997; U.S. Pat. No. 5,888,983 issued Mar. 30, 1999 and Kren et al., 1998, Nature Medicine 4, 285-290), and wherein the sequence part of the disclosed chimeric oligonucleotide complementary to that of the target gene of interest in the above cited document replaces the sequence except the mutator part of the target gene intended to be alterated by the mutator part.

In a preferred embodiment, the present invention comprises a method according to the present invention, wherein step a) is a step of injecting into the tissue containing said cells, or into a tissue adjacent to the patient tissue containing said cells, a composition comprising said nucleic acid.

Also forms part of the present invention, the method according to the present invention, wherein said nucleic acid comprised in the composition is capable of specifically hybridizing with part of target nucleic acid, preferably a target gene (genomic DNA), or target protein belonging to said target cells Among the nucleic acid which can delivered by the method of the present invention, oligonucleotide sens or anti-sens or triple helix capable of modulating the expression products of a target gene of said cells can be cited, in addition to the chimeric oligonucleotides relating to the correction of a functionally deficient gene, or to the creation of a deficient gene disclosed in the above cited documents or in the present specification, as below.

For example, hybridization of antisense oligonucleotides with mRNA can be interfered with the normal functions of mRNA which is protein synthesis.

“Specifically hybridizing” is term which is used to indicate a sufficient degree of complementary such that stable and specific binding occurs between the nucleic acid target, DNA or RNA target, and the nucleic acid which can delivered by the method of the present invention.

In a further preferred embodiment, the invention relates to a method according to the present invention, wherein said nucleic acid, particularly a chimeric oligonucleotide as defined above, comprised in the composition is a polynucleotide containing at least a sequence complementary to a target gene of said cells with the exception of at least one nucleotide which is desired to be inserted, or deleted or substituted in said target gene. “A sequence complementary to a target gene” means a sequence forming in theory Watson-Crick base pairing with part of the target gene sequence, part of the target gene sequence which particularly comprises, in the context of this invention, the fragment of the target sequence wherein said at least one nucleotide is desired to be inserted (or deleted) or changed. Guanine/cytosine or adenine/thymine (or/uracil) are examples of complementary bases which are known to form hydrogen bonds between them.

In a further preferred embodiment, the invention relates to a method according to the present invention wherein said chimeric oligonucleotide comprised in the composition is a chimeric oligonucleotide DNA/2′OMeRNA type designed with two blocks of 2′O-methyl RNA residues flanking a stretch of DNA, poly(T) hairpin loops and a G-C clamp and wherein part of said DNA/2′OMeRNA sequence is complementary to a genomic DNA sequence of a target gene of said cells with the exception of at least single mismatched nucleotide in the DNA stretch when aligned with the target genomic DNA sequence

In a further preferred embodiment, the invention relates to a method according to the present invention wherein said nucleic acid comprised in the composition is a chimeric oligonucleotide DNA/2′OMeRNA type wherein at least part of that DNA/RNA sequence is complementary to a genomic DNA fragment sequence of a target mutated gene of said cells with the exception of that mutation which is desired to be reverted in said target mutated gene.

In a preferred embodiment, said mutation present in the target mutated gene is responsible for an inherited pathology.

In the present specification the term “mutated gene” is understood to mean a gene whose sequence comprises at least one mutation (deletion, addition or substitution of at least one nucleotide) compared to the wild type gene, said mutation being at least partially responsible of a pathology or affection, notably associated with the loss of the normal function of the protein encoded by the wild type functional gene.

In a further preferred embodiment, the invention relates to a method according to the present invention wherein the tissue containing said cells is selected from the group consisting of eye tissues, skeletal muscle tissue, epidermal and dermal tissue.

In a further preferred embodiment, the invention relates to a method according to the present invention, wherein the tissue containing said target cells is selected from the group consisting of eye tissues, and wherein said chimeric oligonucleotide comprised in the composition contains at least a sequence complementary to a genomic DNA sequence of a target gene, said target gene when mutated being at least partially responsible of an eye inherited pathology.

In a more preferred embodiment, the invention relates to a method according to the present invention wherein the eye tissue containing said target cells is retina.

In a further preferred embodiment, the invention relates to a method according to the present invention wherein the tissue containing said target cells is an eye tissue, particularly retina and wherein step a) is a step of intravitreal, periocular (sub conjunctival, peribulbar, laterobulbar, sub tenon), sub-retina or supra choroid injection of the composition comprising said nucleic acid, preferably intravitreal.

Among the target genes which can be chosen in the method of the present invention, gene responsible for inherited retinopathies which are a genetically and phenotypically heterogeneous group of diseases affecting approximately one in 2000 individuals worldwide can be particularly cited (Sohocki et al., Hum Mutat 2001; 17 (1): 42-51).

Among these target genes, the murine gene encoding the cGMP-phosphodiesterase β-subunit wherein the non-sens C→A mutation in the codon 347 of the cDNA of part of said gene leads to retinitis pigmentosa disease, can be cited.

Among these target genes wherein mutations cause retinitis pigmentosa and other inherited retinopathies, the PR1 gene can be particularly cited. Indeed, in that RP1 gene, the missense mutation of the active-site Lys-296 in that rhodopsin gene, such as K296E, has been found to produces an opsin with no chromophore binding site and therefore not activated by light, causing autosomal dominant retinitis pigmentosa (ADRP), or a nonsense mutation R677-STOP has also been found to be associated with retinitis pigmentosa in family linked to the RP1 locus (Payne et al., Invest. Ophthalmol. Vis. Sci., 2000, 41(13):4069-4073; Guillonneau et al., Hum. Mol. Genet., 1999, 8 (8):1541-1546; Pierce et al., Nat. Genet., 1999, 22 (3): 248-254; Li et al., Proc. Natl. Acad. Sci. U S A, 1995, 92 (8); 3551-3555).

Hypoxia inducible factor-1 (HIF-1) is a transcription factor composed of HIF-1 alpha and HIF-1 beta subunits. HIF-1 transactivates multiple genes whose products play key roles in oxygen homeostasis (Ozaki et al., Invest. Ophthalmol, Vis. Sci., 1999, 40 (1): 182-189). So, for example, the gene encoding the transcription factor HIFalpha which governs the expression of several genes involved in inflammation and neovascularization can be targeted to cure patients with ocular neovascularization, mainly retinal neovascularization (Wenger J. Exp. Biol., 2000, 203, 1253-1263), Its normal sequence, PCDHG, is conserved in humans (439-464) and in mice (669-693). A chimeroplast (tems used in the present specification to also designate a chimeric oligonucleotide) bringing a codon stop can be designed in order to have the expressed protein not be able to promote hypoxia induced neovascularization in human or in mice.

So, in a firer preferred embodiment, the invention relates to a method according to the present invention, wherein said chimeric oligonucleotide is a chimeric oligonucleotide DNA/2′OMeRNA type wherein at least part of the sequence of said oligonucleotide is complementary to a genomic DNA sequence fragment of the murine gene encoding the cGMP-phosphodiesterase β-subunit exhibiting the non-sens C→A mutation in the codon 347 of the cDNA of part of said gene leading to retinitis pigmentosa disease, with the exception of that mutated nucleotide A which is replaced by C in said part of the sequence of said oligonucleotide.

In a particularly more preferred embodiment, the invention relates to a method according to the present invention, wherein said chimeric oligonucleotide is selected from the group consisting of:

the chimeric oligonucleotide DNA/2′OMeRNA type having the sequence

SEQ ID No. 1:
CCTTCCAACCTACGTAGCAGAAAGTTTTTACUUUCUGCUACGTAGGUUGG
AAGGGCGCGTTTTCGCGC; and

a DNA/2′OMeRNA type chimeric oligonucleotide sequence of which comprising the essential elements of the sequence SEQ ID No. 1 capable of reverting the non-sens C→A mutation in the codon 347 of the cDNA of the murine gene encoding the cGMP-phosphodiesterase β-subunit in animal, such as a mouse, or human.

In the present specification, the terms “essential elements of the sequence SEQ ID No. 1” means that this sequence contains two blocks of 2′O-methyl RNA residues flanking a stretch of DNA, poly(T) hairpin loops and a G-C clamp and wherein the part of the RNA/DNA sequence which is complementary to that the target gene encoding the cGMP-phosphodiesterase β-subunit can varied and contains the single mismatched nucleotide in the DNA stretch when aligned with the homologous genomic DNA sequence of the functional cGMP-phosphodiesterase β-subunit gene.

In a further preferred embodiment, the invention also relates to a method according to the present invention, wherein said chimeric oligonucleotide is a chimeric oligonucleotide DNA/2′OMeRNA type wherein at least part of the sequence of said oligonucleotide is complementary to a genomic DNA sequence fragment of the murine or human gene encoding the transcription factor HIF1α, with the exception of at least one nucleotide which has been deleted, inserted or substituted in said part of that complementary oligonucleotide, the expressed HIF1α protein coded by the sequence wherein said fragment contains said at least one deleted, inserted or substituted nucleotide being incapable of promoting hypoxia induced neovascularization in human or in mouse.

In a particularly more preferred embodiment, said oligonucleotide of the DNA/2′OMeRNA type chimeric oligonucleotide complementary to a genomic DNA sequence fragment of the mouse or human gene encoding the transcription factor HIF1α is selected from the group consisting of:

an oligonucleotide capable of inducing the mutation B142-STOP in the murine or human transcription factor HIF1α, and

the oligonucleotide having the sequence SEQ ID No. 2; CCA TGT GAC CAT TAG GAA ATG AGA G, or an oligonucleotide comprising a fragment thereof capable of inducing the same mutation.

The normal part of the sequence of the murine HIF1α gene wherein the mutation E142-STOP can be induced is: CCA TGT GAC CAT GAG GAA ATG AGA G (SEQ ID No. 7). The chimeroplast capable of inducing that mutation B142-STOP in human or in mouse is named Chi H/M E142-STOP (“Chi” for Chimeroplast, “H” for Human, “M” for mouse).

In a further preferred embodiment, the invention also relates to a method according to the present invention, wherein said chimeric oligonucleotide is a chimeric oligonucleotide DNA/2′OMeRNA type wherein at least part of the sequence of said oligonucleotide is complementary to a genomic DNA sequence fragment of the murine or human RP1 gene, with the exception of at least one nucleotide which has been deleted, inserted or substituted in said part of that complementary oligonucleotide.

In a particularly more preferred embodiment, said oligonucleotide of the DNA/2′OMeRNA type chimeric oligonucleotide complementary to a genomic DNA sequence fragment of the murine or human RP1 gene is selected from the group consisting of:

an oligonucleotide capable of reverting the mutation K296E or R677-STOP in the human RP1 protein;

the oligonucleotide having the sequence SEQ ID No. 3: GCT TTC TTT GCC AAG AGC GCC GCA or an oligonucleotide comprising a fragment thereof capable of reverting the same mutation K296E; and

the oligonucleotide having the sequence SEQ ID No 4: AAG AAA AAA TCT AGA CAA GCA A or an oligonucleotide comprising a fragment thereof capable of reverting the same mutation R677-STOP.

The part of the sequence of the RP1 mutated human gene wherein that mutation K296E can be reverted to correct the opsin mutation in human is: GCT TTC TTT GCC GAG AGC GCC GCA (SEQ ID No. 8).

The chimeroplast capable of reverting that mutation K296E in human is named Chi HOPS E296K (“OPS” for opsin).

The part of the sequence of the RP1 mutated human gene wherein that mutation R677-STOP can be reverted to correct the RP1 mutation in human is: AAG AAA AAA TCT TGA (SEQ ID No. 9).

The chimeroplast capable of reverting that mutation R677-STOP in human is named Chi HRP1 R677-STOP.

In a particularly more preferred embodiment, said oligonucleotide of the DNA/2′OMeRNA type chimeric oligonucleotide complementary to a genomic DNA sequence fragment of the murine RP1 gene is selected from the group consisting of:

an oligonucleotide sequence capable of inducing the mutation K296E or E348-STOP in the murine RP1 protein sequence;

the oligonucleotide having the sequence SEQ ID No. 5: GCT TTC TTT GCT GAG AGC TCT TCC A

or an oligonucleotide comprising a fragment thereof capable of reverting the same mutation K296E; and

the oligonucleotide having the sequence SEQ ID No. 6: AAG ACT TCT GAG TAA CAA TCA A or an oligonucleotide comprising a fragment thereof capable of reverting the same mutation E348-STOP.

These chimeroplasts “Chi MOPSK296 E”, designed to induce a very handful mutation in opsin in a mice, and “Chi MPR1 E348-STOP” can be used to create a model of mutation in mice, notably a model of retinal degeneration according to previous knowledge that the mutation of opsin in human leads to a rapid retinal degeneration.

The normal part of the sequence of the RP1 gene in mice wherein the mutation K296E can be induced is: GCT TTC TTT GCT AAG AGC TCT TCC A (SEQ ID No. 10).

Devices for transdermal, transcutaneous delivery of therapeutic agents through iontophoresis are commonly used for treating skin or eye diseases, and thus have been already disclosed. So, the skilled artisan could easily choose and determined the iontophoresis device and its use conditions, particularly the current density, the period of time of applying the current and the electrodes form and location etc., adapted to the tissue containing the target cells where the nucleic acid transfer is desired to be done.

Among the iontophoresis devices which have been already disclosed, the devices disclosed in the following patent documents can be cited: U.S. Pat. No. 4,141,359 issued Feb. 27, 1979; U.S. Pat. No. 4,250,878 issued Jan. 17, 1981; U.S. Pat. No. 4,301,794 issued Nov. 24, 1981; U.S. Pat. No. 4,747,819 issued Apr. 31, 1988; U.S. Pat. No. 4,752,285 issued Jun. 21, 1988; U.S. Pat. No. 4,915,685 issued Apr. 10, 1990; U.S. Pat. No. 4,979,938 issued Dec. 25, 1990; U.S. Pat. No. 5,252,022 issued Oct. 5, 1993; U.S. Pat. No. 5,374,245 issued Dec. 20, 1994; U.S. Pat. No. 5,498,235 issued Mar. 12, 1996; U.S. Pat. No. 5,730,716 issued Mar. 24, 1998; U.S. Pat. No. 6,001,088 issued Dec. 14, 1999; U.S. Pat. No. 6,018,679 issued Jan. 25, 2000; U.S. Pat. No. 6,139,537 issued Oct. 31, 2000; U.S. Pat. No. 6,148,231 issued Nov. 14, 2000; U.S. Pat. No. 6,154,671 issued Nov. 28, 2000 and U.S. Pat. No. 6,167,302 issued Dec. 26, 2000, documents which are herein incorporated by reference.

Among the iontophoresis devices which can be used for intraocular delivery of nucleic acid, particularly chimeric oligonucleotide as defined above (“ocular chimeraplasty”), in the method according to the present invention, the iontophoresis system disclosed in the patent document U.S. Pat. No. 6,154,671 issued Nov. 28, 2000, is preferred for step b) of the method.

That above cited device (disclosed in the patent document U.S. Pat. No. 6,154,671) particularly comprises a reservoir configured to receive the composition comprising said nucleic acid, in case said nucleic acid is topically applied or injected potentially in an ionized solution in step a), or an aqueous solution, in case said nucleic acid is injected in step a), and having an internal wall, an external wall, and an end wall bridging the internal wall and the external wall, the internal wall and the external wall being annular and having a free end configured to be applied to an eyeball, said device further comprising at least one active electrode arranged in the reservoir, another electrode and a current generator, wherein the at least one electrode is a surface electrode arranged on an interior surface of the end wall and wherein the internal wall has an outer diameter that is configured to be at least equal to a predetermined diameter, whereby the predetermined diameter represents a diameter of a human cornea.

In another aspect, the present invention is directed to a method to treat a disease comprising the administration of an acid nucleic, preferably a chimeric oligonucleotide as defined above, capable of reverting or inducing a mutation in a target gene of target cells, gene expression of which is associated to that disease, in a human or animal host in need of such treatment, wherein the method used for delivering in vivo said nucleic acid into said target cells is the method for delivering in vivo nucleic acid according to the present invention.

In a preferred embodiment, in the method to treat a disease according to the invention, said disease is an inherited pathology.

In a more preferred embodiment, in the method to treat a disease according to the invention, said disease an inherited retinopathy.

In another aspect, the present invention is directed to a method to obtain an animal model comprising the administration of an acid nucleic, preferably a chimeric oligonucleotide as defined above, capable of reverting or inducing a mutation in a target gene of target cells of that animal, wherein the method used for delivering in vivo said nucleic acid into said target cells is the method for delivering in vivo nucleic acid according to the present invention.

In another aspect, the present invention is directed to a method for the screening of pharmaceutical or cosmetic compounds comprising the use of an animal model, a target gene of target cells of which has been modified by the administration of an acid nucleic, preferably a chimeric oligonucleotide as defined above, a chimeric oligonucleotide capable of reverting or inducing a mutation in that target gene, wherein the method used for delivering in vivo said nucleic acid into said target cells is the method for delivering in vivo nucleic acid according to the present invention.

In another different aspect, the present invention is directed to a chimeric oligonucleotide DNA/2′OMeRNA type designed with two blocks of 2′O-methyl RNA residues flanking a stretch of DNA, poly(T) hairpin loops and a G-C clamp and wherein part of said DNA/2′OMeRNA sequence is complementary to a genomic DNA sequence of a target gene of said cells with the exception of at least single mismatched nucleotide in the DNA stretch when aligned with the target genomic DNA sequence, characterized in that said at least part of the sequence complementary to that target gene is selected from the group consisting of

an oligonucleotide sequence capable of reverting the non-sens C→A mutation in the codon 347 of the cDNA of the murine gene encoding the cGMP-phosphodiesterase β-subunit.

A chimeric oligonucleotide DNA/2′OMeRNA type according to claim 27 having the sequence SEQ ID No. 1 is preferred.

In another aspect, the present invention is directed to a chimeric oligonucleotide DNA/2′OMeRNA type designed with two blocks of 2′O-methyl RNA residues flanking a stretch of DNA, poly(T) hairpin loops and a G-C clamp and wherein part of said DNA/2′OMeRNA sequence is complementary to a genomic DNA sequence of a target gene of said cells with the exception of at least single mismatched nucleotide in the DNA stretch when aligned with the target genomic DNA sequence, characterized in that said at least part of the sequence complementary to that target gene is selected from the group consisting of:

an oligonucleotide sequence capable of inducing a nonsense mutation STOP in the DNA encoding the murine or human transcription factor HIF1 α so that the protein expressed by such a mutated HIF1α gene is not functional;

an oligonucleotide sequence capable of inducing the mutation E142-STOP in the protein coded by the mouse transcription factor HIF 1α, or the corresponding mutation in the human HIF1α protein sequence;

the oligonucleotide sequence having the sequence SEQ ID No. 2, or an oligonucleotide comprising a fragment thereof capable of inducing the same mutation.

In another aspect, the present invention is directed to a chimeric oligonucleotide DNA/2′OMeRNA type designed with two blocks of 2′O-methyl RNA residues flanking a stretch of DNA, poly(T) hairpin loops and a G-C clamp and wherein part of said DNA/2′OMeRNA sequence is complementary to a genomic DNA sequence of a target gene of said cells with the exception of at least single mismatched nucleotide in the DNA stretch when aligned with the target genomic DNA sequence, characterized in that said at least part of the sequence complementary to that target gene is selected from the group consisting of

an oligonucleotide sequence capable of reverting a mutation in the DNA encoding the human RP1 protein, said mutation being responsible for the expression of a non-functional protein, RP1 or opsin protein;

an oligonucleotide sequence capable of reverting the mutation K296E or R677-STOP in the human opsin or RP1 protein sequence respectively;

the oligonucleotide having the sequence SEQ ID No. 3 or an oligonucleotide comprising a fragment thereof capable of reverting the same mutation K296E; and

the oligonucleotide having the sequence SEQ ID No. 4 or an oligonucleotide comprising a fragment thereof capable of reverting the same mutation R677-STOP.

In another aspect, the present invention is directed to a chimeric oligonucleotide DNA/2′OMeRNA type designed with two blocks of 2′O-methyl RNA residues flanking a stretch of DNA, poly(T) hairpin loops and a G-C clamp and wherein part of said DNA/2′OMeRNA sequence is complementary to a genomic DNA sequence of a target gene of said cells with the exception of at least single mismatched nucleotide in the DNA stretch when aligned with the target genomic DNA sequence, characterized in that said at least part of the sequence complementary to that target gene is selected from the group consisting of:

an oligonucleotide sequence capable of inducing a mutation in the DNA encoding the murine RP1 protein, said mutation being responsible for the expression of a non-functional protein, RP1 or opsin protein;

an oligonucleotide sequence capable of inducing the mutation K296E or E348-STOP in the murine opsin or RP1 protein sequence respectively;

the oligonucleotide having the sequence SEQ ID No. 5 or an oligonucleotide comprising a fragment thereof capable of reverting the same mutation K296E; and

the oligonucleotide having the sequence SEQ ID No. 6 or an oligonucleotide comprising a fragment thereof capable of reverting the same mutation E348-STOP.

In another different aspect, the present invention is directed to a pharmaceutical composition comprising a chimeric oligonucleotide DNA/2′OMeRNA type according to the present invention.

In another different aspect, the present invention is directed to a method to treat a human host having a retinopathy induced by the presence of a mutation in the PRI gene, comprising contacting in vivo the host PRI genomic DNA with the chimeric oligonucleotide DNA/2′OMeRNA capable of reverting the mutation K296E or R677-STOP in the human opsin or RP1 protein sequence respectively according to the present invention.

In another different aspect, the present invention is directed to a method to treat a human or an animal host having ocular neovascularization induced by the expression of the normal transcription factor HIF1α gene, comprising contacting in vivo the host HIF1α genomic DNA with the chimeric oligonucleotide DNA/2′OMeRNA sequence capable of inducing a nonsense mutation STOP in the DNA encoding the human or murine transcription factor HIF1α according to the present invention, so that the protein expressed by such a mutated HIF1α human or animal gene is not functional.

In another different aspect, the present invention is directed to an animal model comprising a mutation in the RP1, mutation which has been induced by the in vivo or ex vivo administration of a chimeric oligonucleotide wherein said chimeric oligonucleotide is a chimeric oligonucleotide capable of inducing a RP1 mutation according to the present invention.

The use of an animal model according to the present invention for the screening of pharmaceutical compounds capable of treating human or animal retinopathies forms also part of the invention.

To assist in understanding the present invention, the following examples are included which describe the results of a series of experiments. These examples relating to the present invention are illustrative and should not, of course, be constructed as specifically limiting the invention. Moreover, such variations of the invention, now known or later developed, which would be within the purview of one skilled in the art are to be considered to fall within the scope of the present invention hereinafter claimed.

### EXAMPLE I

### Treatment of the Retinal Degeneration of the rd Mouse by Iontopherically Transferring in vivo a Chimeric Oligonucleotide into Retina Cells

I: Molecular Basis of the Retinal Degeneration in rd Mice

Mice homozygous for the rd mutation display hereditary retinal degeneration and serve as a model for human retinitis pigmentosa. In affected animals, the retinal rod photoreceptor cells begin degenerating at about postnatal day 8 and by four weeks no cones are left Degeneration is preceded by accumulation of cyclic GMP in the retina and is correlated with deficient activity of the rod cGMP-phosphodiesterase. This enzymatical defect is due to the presence of a nonsense C→A mutation in the rd β-PDE gene. The nonsense mutation creates an ochre stop codon (position 347) within exon 7 and leads to the truncation of the resulting cGMP-phosphodiesterase β-subunit. The absence of a functional cGMP-phosphodiesterase protein in rd/rd mice is responsible for retinal degeneration.

It can be assumed that a revertion of the stop mutation of the rd β-PDE gene will lead to a functional protein in the photoreceptor and the disease is cured. The strategy using chimeric oligonucleotides, proved to be efficient in other models of hereditary diseases due to point mutations, has been chosen for this challenge. So, the chimeraplasty has been used to correct the nonsense mutation responsible for the retinal degeneration of the rd mouse.

The chimeric oligonucleotides were delivered into the targeted tissue using the combination of both, local injection and iontophoresis.

### EXAMPLE II

### Materials and Methods

### Materials

1) Chimeric Oligonucleotides

The DNA/2′OMeRNA chimeric oligonucleotides were synthetisized and purified by high pressure liquid chromatography by GensetOligos (France). The oligonucleotides were resuspended in distilled water and quantitated by ultra-violet absorbance at 260 nm. The sequences of the chimeric oligonucleotides were are follows:

Specific chimeric oligonucleotide (named Chi) having the following sequence (sequence SEQ ID No. 1) (the 2′OMeRNA nucleotides are underlined):

5′CCTTCCAACCTACGTAGCAGAAAGTTTTTACUUUCUGCUACGTAGGUU
GGAAGGGCGCGTTTTCGCGC3′

Control chimeric oligonucleotide (named Ctr) (sequence SEQ ID No. 11) (the 2′OMe RNA nucleotides are underlined):

5′CTACCAAATCCATGGGATTTCCATCAGTTAUUUCUGUCCATCAGGUAG
GAGUGGGCTCGCGTGCGTTC3′

2)Animals

C3H/HeN mice with a nonsense mutation (position 347) were purchased (Iffa Credo). Genotyping to verify the absence or presence of the rd/rd mutation was accomplished by PCR of DNA from tail biopsies and subsequent restriction fragment analysis. The animals were given food and water ad libilum and maintained under pathogen-free conditions of 12 h-light/12 h darkness.

3) Coulomb-controlled Iontophoresis (CCI) System

Iontophoresis was performed using the drug delivery device designed by OPTIS France (as disclosed in the U.S. Pat. No. 6,154,671 dated Nov. 18, 2000) A container was designed to allow transcorneoscleral iontophoresis. A platinium electrode was placed at the bottom of the container and two silicone tubes were settled laterally, One tube was used to infuse saline buffer and the other to aspirate bubbles. The CCI electronic unit can delivered up to 2,500 μA for 600 sec. An audio-visual alarm indicated each disruption in the electric circuit ensuring a calibrated and controlled delivery of the product. To proceed with the iontophoresis treatment, the CCI ocular cup was placed on the eye and the other electrode was maintained in contact with the animal.

### Methods

1) Injection and Iontophoresis

The experiments were conducted in accordance with the ARVO statement for the use of animals in ophtalmic and vision research. The following treatment was administered on postnatal day (P) 7 and repeated on P9: Mice were anesthetized with an intra-pertoneal injection of chlorpromazine and ketamin. Ocular injections were performed into the vitreous using a glass micro-capillary under microscopic visualization. Just after intravitreal injection, coulomb controlled iontophoresis was performed. The iontophoresis parameters were 300 μA for 300 sec. The negatively charged electrode was placed onto the eye. A solution of phosphate buffered saline (PBS) was continuously pumped into the drug container.

2) Oligonucleotide Transfection Analysis using a Biotinylated Chimeric Oligonucleotid

Biotinylated chimeric oligonucleotide were injected and followed by iontophoresis as described above. The eyes were enucleated 1 h after the treatment, immediately frozen in OCT (Tissue Tek, USA) and sectioned (10 μm). They were fixed in methanol at −20° C. for 10 min. The sections were then washed in 1% Triton X-100 PBS and incubated in a 1/100 streptavidin horseradish-peroxidase PBS solution for 2 h at room-temperature. The sections were washed and the complex was revealed using 3.3′diaminobenzidine tetrahydrochloride in the presence of H₂O₂. Finally, the sections were counterstained with Hemalun.

3) rd-mutation Test by Restriction Fragment Analysis of RT-PCR Products

Total RNA was extracted from single retina of rd/rd mice 18 days after the last treatment (P27) by the acid guanidinium thiocyanate-phenol-chloroform method. Retinal total RNA (1 μg) was used as template to synthesize cDNA in a volume of 20 μl using 200U of Moloney leukemia virus (MLV) reverse transcriptase (10 min at 21° C., 1 at 42° C,. 5 min at 55° C., 10 min at 42° C.). Oligonucleotide primers included the sequences 5′-GGCCGGGAAATTGTCTTCTAC-3′ (sequence SEQ ID No. 12) and 5′-CCCCAGGAACTGTGTCAGAGA-3′(sequence SEQ ID No. 13), located at nucleotide positions 921 to 943 and 1258 to 1279 of the β-cGMP-phosphodiesterase cDNA respectively. RT product was amplified by PCR in a volume of 100 μl using 3U of Taq polymerase and primers described above, 30 PCR cycles were performed in thermal cycler with an initial denaturation of 5 min at 94° C., denaturation temperature of 94° C. for 1 min, annealing temperature of 55° C. for 1 min, extension temperature of 72° C. for 1 min and a final extension of 10 min at 72° C. The PCR buffer contained α-³²P dCTP. After each PCR reaction, products were digested with 2.5 units BsaAI and/or 5 units DdeI in the provided buffer at 37° C. overnight, then ethanol precipitated, washed and resuspended in 10 μl gel loading buffer. The products were run on an 8% nondenaturing polyacrylamyde gel at 500 volts for 3 hours. The gel was exposed to a film for 3 days. RNA from +/+ retinae and from untreated rd/rd retinae served as controls.

4) Immunohistochemistry of flat-mounted Retinas

To analyze the survival rate of rod-photoreceptors in control and control treated animals, we performed opsin-immunohistochemistry on whole-mounted retina. Our antibody Rho4D2 recognises specifically opsin, which is the photo-pigment of rod-photoreceteptors. Eyes were enucleated and fixed for 30 min in PBS/Paraformaldehyde 4%. The retinae were dissected and fixed in methanol at −20° C. for 10 min, washed three times in 1% Triton X-100 PBS, incubated over-night in a 1/100 Rho4D2, 1% Triton X-100 PBS solution at room temperature. The retina were then washed, incubated for 2 h at room temperature with an 1/250 anti-mouse Alexa 40 antibody, washed and flat-mounted in glycerol/PBS. They were viewed and photographed by fluorescence microscopy (see FIG. 3B). The photographs were all taken with the same film (Illford 400ASA), exposure time (1 h 30 min), and developed in exactly the same manner. The photographs of flat-mounted retinae were scanned. The number of rod-photoreceptors were measured using a computerized image-analysis system (NIH)

5) Statistical Analysis

Results were expressed as mean i standard error of the mean (SEM) (see FIG. 3A). Statistical analyses were performed using the the non parametric Man Whitney U test.

### EXAMPLE III

### Results and Discussion

### Chimeraplast Design

Using chimeraplasty rules, a DNA/RNA2′OMe oligonucleotide (named Chi) has been designed which has the potentialities to revert the C→A point mutation located within codon 347 in the mouse rd β-PDE gene. A control oligonucleotide (named Ctr) contains the same base composition as the active chimeric oligonucleotide but a different sequence.

### Photoreceptor Transfection by Chimeric Oligonucleotides

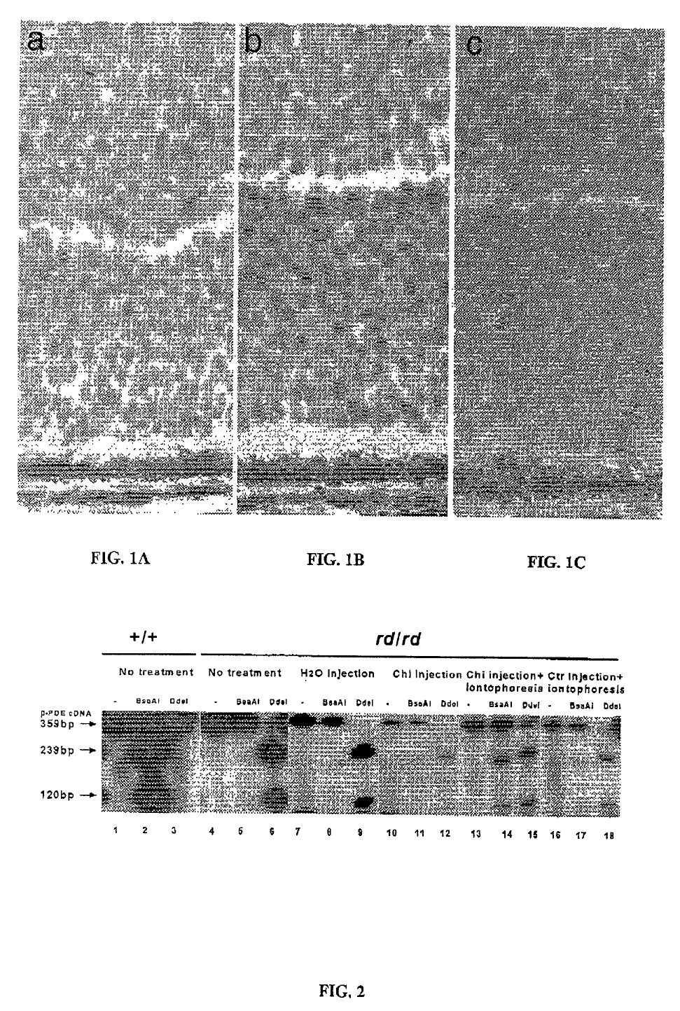

The experiments with the biotinylated oligonucleotide clearly demonstrate that iontophoresis enhances the oligonucleotide uptake in retinal cells, compared to intravitreal injections only as. Notably the uptake in photoreceptors is clearly visible in FIGS. 1A to 1C.

### Point Mutation Correction within rd β-PDE mRNA

RT-PCR were performed with rd β-PDE mRNA specific primers on extracted retinae. The rd nonsense point mutation in codon 347 creates a DdeI restriction site and removes a BsaAI site from the wild-type sequence. Digesting the 359 bp β-PDE cDNA with BsaAI or DdeI yields two diagnostic fragments of 120 bp and 239 bp. This method allows the differenciation of the mutated sequence (DdeI sensitive) from the wild-type one (BsaAI sensitive) at the mRNA level.

Intravitreal injection and iontophoresis were performed on postnatal day 7 and 9 mice. RT-PCR experiments followed by restriction digestions were performed in different conditions in order to check the effect of the chimeric oligonucleotide on the rd β-PDE mRNA correction at postnatal day 27.

The gel in FIG. 2 showed that:

The rd/rd β-PDE cDNA were totally cut only by DdeI, which recognized only the mutated sequence (lanes 4-6);

the +/+ β-PDE cDNA were totally cut by BsaAI, which recognized only the wild type sequence (lanes 1-3). Nevertheless, the slight presence of BsaAI digestion product indicated a slight lack of specificity of BsaAI;

The β-PDE cDNA from chimeraplast-traited mice were cut by BsaAI and partly by DdeI only if the intravitreal injection was followed by iontophoresis (lanes 13-15). It demonstrated that the chimeric oligonucleotide Chi could revert the rd point mutation into the wild- type nucleotide. Moreover it indicated that only the combination of both techniques (intravitreal injection and iontophoresis) could allow chimeraplast-mediated gene correction;

The β-PDE cDNA from control chimeraplast-traited mice were cut by Ddel and slightly by BsaAI (lanes 16-18). The BsaAI reactivity could be explained by its lack of specificity already observed after rd/rd W-PDE cDNA digestion (lane 5); and

The β-PDE cDNA from water-traited mice were cut only by DdeI showing that gene correction occurs only in the presence of chimeric oligonucleotides (lanes 7-9).

### Photoreceptor Rescue

The amount of rod-photoreceptors was counted on flat-mounted retina of chimeraplast treated animals and control at P27. In untreated animals and control treated animals, as well as in animals treated with a intravitreal water-injection followed by iontophoresis, the survival at that stage of the disease is negligible. A highly significant increase in rod-photoreceptor-survival can be observed in chimeraplast/iontophoresis treated animals only. iontophoresis is known to be a non-invasive process to deliver drugs using a low-intensity current. It uses an electrode of the same polarity as the charge on the drug to drive ionic drugs into the tissues. The present inventors have so demonstrated that iontophoresis can be used to enhance the nucleic acid penetration into cells tissue, such as chimeric oligonucleotide DNA/2′OMeRNA type, particularly into ocular cells after intra- or peri-ocular injection and to enhance retinal transfer or penetration after or before or simultaneously to intraocular injection.

We claim:

1. A method for delivering in vivo a chimeric oligonucleotide into target cells of an animal or human tissue, comprising the steps of:
a) topically applying to or injecting into the patient tissue, or a patient tissue adjacent to the patient tissue containing said target cells, a composition comprising said chimeric oligonucleotide; and
b) transferring said chimeric oligonucleotide into said target cells by iontophoresis.

2. The method of claim 1, wherein step b) is carried out prior to, during or after the step a).

3. The method of claim 1, wherein step a) is a step of injecting said composition.

4. The method of claim 1, wherein said chimeric oligonucleotide comprised in the composition is capable of specifically hybridizing with a sequence of a genomic DNA contained in said target cells.

5. The method of claim 1, wherein said chimeric oligonucleotide comprised in the composition is a chimeric oligonucleotide capable of modified the expression products of a target gene of said target cells.

6. The method of claim 1, wherein said chimeric oligonucleotide comprised in the composition is an oligonucleotide containing at least a sequence complementary according to Watson-Cricks rules to a target gene of said cells with the exception of at least one nucleotide which is desired to be inserted, deleted or substituted in said target gene.

7. The method of claim 1,.wherein said chimeric oligonucleotide comprised in the composition is a chimeric oligonucleotide DNA/2′OMeRNA type designed with two blocks of 2′O-methyl RNA residues flanking a stretch of DNA, poly(T) hairpin loops and a G-C clamp and wherein part of said DNA/2′OMeRNA sequence is complementary to a genomic DNA sequence of a target gene of said cells with the exception of at least single mismatched nucleotide in the DNA stretch when aligned with the target genomic DNA sequence.

8. The method of claim 7, wherein said chimeric oligonucleotide comprised in the composition is a chimeric oligonucleotide DNA/2′OMeRNA wherein at least part of that DNA/RNA sequence is complementary to a genomic DNA sequence of a target mutated gene of said cells, said mutation being responsible for an inherited pathology, with the exception of that mutation which is desired to be reverted in said target gene.

9. The method of claim 1, wherein the tissue containing said target cells is selected from the group consisting of eye tissues, skeletal muscle tissue, epidermal and dermal tissue.

10. The method of claim 1, wherein the tissue containing said target cells is selected from the group consisting of eye tissues and wherein said chimeric oligonucleotide comprised in the composition contains at least a sequence complementary to a genomic DNA sequence of a target gene, said target gene when mutated being at least partially responsible of an eye inherited pathology.

11. The method of claim 8, wherein the eye tissue containing said cells is retina.

12. The method of claim 8, wherein step a) is a step of intraviteal injection of the composition comprising said chimeric oligonucleotide.

13. The method of claim 1, wherein said chimeric oligonucleotide is a chimeric oligonucleotide DNA/2′OMeRNA type wherein at least part of the sequence of said oligonucleotide is complementary to a genomic DNA sequence fragment of the murine gene encoding the cGMP-phosphodiesterase β-subunit exhibiting the non-sens C→A mutation in the codon 347 of the cDNA of part of said gene leading to retinitis pigmentosa disease, with the exception of that mutated nucleotide A which is replaced by C in said part of the sequence of said oligonucleotide.

14. The method of claim 13, wherein said chimeric oligonucleotide is selected from the group consisting of:
the chimeric oligonucleotide DNA/2′OMeRNA type having the sequence SEQ ID No. 1; and
a DNA/2′OMeRNA type chimeric oligonucleotide sequence of which comprising the essential elements of the sequence SEQ ID No. 1 capable of reverting the non-scns C→A mutation in the codon 347 of the cDNA of the murine gene encoding the cGMP-phosphodiesterase β-subunit in animal or human.

15. The method of claim 1, wherein said chimeric oligonucleotide is a chimeric oligonucleotide DNA/2′OMeRNA type wherein at least part of the sequence of said oligonucleotide is complementary to a genomic DNA sequence fragment of the mouse or human gene encoding the transcription factor HIF1α, with the exception of at least one nucleotide which has been deleted, inserted or substituted in said part of that complementary oligonucleotide, the expressed HIF1α protein coded by the sequence wherein said fragment contains said at least one deleted, inserted or substituted nucleotide being incapable of promoting hypoxia induced neovascularization in human or in mouse.

16. The method of claim 15, wherein said complementary oligonucleotide of the DNA/2′OMeRNA type chimeric oligonucleotide is selected from the group consisting of:
an oligonucleotide capable of inducing the mutation E142-STOP in the murine or human transcription factor HIF1α; and
the oligonucleotide having the sequence SEQ ID No. 2 or an oligonucleotide comprising a fragment thereof capable of inducing the same mutation.

17. The method of claim 1, wherein said chimeric oligonucleotide is a chimeric oligonucleotide DNA/2′OMeRNA type wherein at least part of the sequence of said oligonucleotide is complementary to a genomic DNA sequence fragment of the murine or human RP1 gene, with the exception of at least one nucleotide which has been deleted, inserted or substituted in said part of that complementary oligonucleotide.

18. The method of claim 17, wherein said complementary oligonucleotide of the DNA/2′OMeRNA type chimeric oligonucleotide is selected from the group consisting of
an oligonucleotide capable of reverting the mutation K296E or R677-STOP in the human RP1 protein;
the oligonucleotide having the sequence SEQ ID No. 3 or an oligonucleotide comprising a fragment thereof capable of reverting the same mutation K296E; and
the oligonucleotide having the sequence SEQ ID No. 4 or an oligonucleotide comprising a fragment thereof capable of reverting the same mutation R677-STOP.

19. The method of claim 17, wherein said complementary oligonucleotide of the DNA/2′OMeRNA type chimeric oligonucleotide is selected from the group consisting of;
an oligonucleotide sequence capable of inducing the mutation K296E or E348-STOP in the murine RP1 protein sequence;
the oligonucleotide having the sequence SEQ ID No. 5 or an oligonucleotide comprising a fragment thereof capable of reverting the same mutation K296E; and
the oligonucleotide having the sequence SEQ ID No. 6 or an oligonucleotide comprising a fragment thereof capable of reverting the same mutation E348-STOP.

20. The method of claim 1, wherein the iontophoresis system used in step b) is a device selected in the group consisting of the devices disclosed in the following patents: U.S. Pat. No. 4,141,359 issued Feb. 27, 1979; U.S. Pat. No. 4,250,878 issued Jan. 17, 1981; U.S. Pat. No. 4,301,794 issued Nov. 24, 1981; U.S. Pat. No. 4,747,819 issued Apr. 31, 1988; U.S. No. Pat. 4,752,285 issued Jun. 21, 1988; U.S. Pat. No. 4,915,685 issued Apr. 10, 1990; U.S. Pat. No. 4,979,938 issued Dec. 25, 1990; U.S. Pat. No. 5, 252, 022 issued Oct. 5, 1993; U.S. Pat. No. 5,374,245 issued Dec. 20, 1994; U.S. Pat. No. 5,498,235 issued Mar. 12, 1996; U.S. Pat. No. 5,730,716 issued Mar. 24, 1998; U.S. Pat. No. 6,001,088 issued Dec. 14, 1999; U.S. Pat. No. 6,018,679 issued Jan. 25, 2000, U.S. Pat. No. 6,139,537 issued Oct. 31, 2000; U.S. Pat. No. 6,148,231 issued Nov. 14, 2000; U.S. Pat. No. 6,154,671 issued Nov. 28, 2000, and U.S. Pat. No. 6,167,302 issued Dec. 26, 2000.

21. The method of claim 20, wherein the iontophoresis system used in step b) is a device selected in the group consisting of the devices disclosed in the U.S. Pat. No. 6,154,671 issued Nov. 28, 2000.

22. A method to treat a disease comprising the administration of a chimeric oligonucleotide capable of reverting or inducing a mutation in a target gene of target cells, gene expression of which is associated to that disease, in a human or animal host in need of such treatment, wherein the method used for delivering in vivo said chimeric oligonucleotide into said target cells is the method according to claim 1.

23. The method to treat a disease according to claim 22, wherein said disease is an inherited pathology.

24. The method to treat a disease according to claim 22, wherein said disease is an inherited retinopathy.

25. A method to obtain an animal model comprising the administration of a chimeric oligonucleotide capable of reverting or inducing a mutation in a target gene of target cells of that animal, wherein the method used for delivering in vivo said chimeric oligonucleotide into said target cells is the method according to claim 1.

26. A method for the screening of pharmaceutical or cosmetic compounds comprising the use of an animal model, a target gene of target cells of which has been modified by the administration of a chimeric oligonucleotide capable of reverting or inducing a mutation in that target gene, wherein the method used for delivering in vivo said chimeric oligonucleotide into said target cells is the method according to claim 1.

27. A chimeric oligonucleotide DNA/2′OMeRNA type designed with two blocks of 2′O-methyl RNA residues flanking a stretch of DNA, poly(T) hairpin loops and a G-C clamp and wherein part of said DNA/2′OMeRNA sequence is complementary to a genomic DNA sequence of a target gene of said cells with the exception of at least single mismatched nucleotide in the DNA stretch when aligned with the target genomic DNA sequence, characterized in that said at least part of the sequence complementary to that target gene is selected from the group consisting of:
an oligonucleotide sequence capable of reverting the non-sens C→A mutation in the cod on 347 of the cDNA of the murine gene encoding the cGMP-phosphodiesterase β-subunit.

28. The chimeric oligonucleotide DNA/2′OMeRNA type according to claim 27 having the sequence SEQ ID No. 1.

29. A chimeric oligonucleotide DNA/2′OMeRNA type designed with two blocks of 2′O-methyl RNA residues flanking a stretch of DNA, poly(T) hairpin loops and a G-C clamp and wherein part of said DNA/2′OMeRNA sequence is complementary to a genomic DNA sequence of a target gene of said cells with the exception of at least single mismatched nucleotide in the DNA stretch when aligned with the target genomic DNA sequence, characterized in that said at least part of the sequence complementary to that target gene is selected from the group consisting of:
an oligonucleotide sequence capable of inducing a nonsense mutation STOP in the DNA encoding the murine or human transcription factor HIF1α so that the protein expressed by such a mutated HIF1α gene is not functional;
an oligonucleotide sequence capable of inducing the mutation E142-STOP in the protein coded by the mouse transcription factor HIF1α, or the corresponding mutation in the human HIF1α protein sequence;
the oligonucleotide sequence having the sequence SEQ ID No. 2 or an oligonucleotide comprising a fragment thereof capable of inducing the same mutation.

30. A chimeric oligonucleotide DNA/2′OMeRNA type designed with two blocks of 2′O-methyl RNA residues flanking a stretch of DNA, poly(T) hairpin loops and a G-C clamp and wherein part of said DNA/2′OMeRNA sequence is complementary to a genomic DNA sequence of a target gene of said cells with the exception of at least single mismatched nucleotide in the DNA stretch when aligned with the target genomic DNA sequence, characterized in that said at least part of the sequence complementary to that target gene is selected from the group consisting of:
an oligonucleotide sequence capable of reverting a mutation in the DNA encoding the human RP1 protein, said mutation being responsible for the expression of a non-functional protein;
an oligonucleotide sequence capable of reverting the mutation K296E or R677-STOP in the human opsin or RP1 protein sequence;
the oligonucleotide having the sequence SEQ ID No. 3 or an oligonucleotide comprising a fragment thereof capable of reverting the same mutation K296E; and
the oligonucleotide having the sequence SEQ ID No. 4 or an oligonucleotide comprising a fragment thereof capable of reverting the same mutation R677-STOP.

31. A chimeric oligonucleotide DNA/2′OMeRNA type designed with two blocks of 2′O-methyl RNA residues flanking a stretch of DNA, poly(T) hairpin loops and a G-C clamp and wherein part of said DNA/2′OMeRNA sequence is complementary to a genomic DNA sequence of a target gene of said cells with the exception of at least single mismatched nucleotide in the DNA stretch when aligned with the target genomic DNA sequence, characterized in that said at least part of the sequence complementary to that target gene is selected from the group consisting of:
an oligonucleotide sequence capable of inducing a mutation in the DNA encoding the murine RP1 protein, said mutation being responsible for the expression of a non-functional protein;
an oligonucleotide sequence capable of inducing the mutation k296E or E348-STOP in the murine opsin or RP1 protein sequence;
the oligonucleotide having the sequence SEQ ID No. 5 or an oligonucleotide comprising a fragment thereof capable of reverting the same mutation K296E; and
the oligonucleotide having the sequence SEQ ID No. 6 or an oligonucleotide comprising a fragment thereof capable of reverting the same mutation E348-STOP.

32. A pharmaceutical composition comprising a chimeric oligonucleotide DNA/2′OMeRNA type of claims 27 to 30.

33. A method to treat a human host having a retinopathy induced by the presence of a mutation in the PR1 gene, comprising contacting in vivo the host PR1 genomic DNA with the chimeric oligonucleotide DNA/2′OMeRNA of claim 30.

34. A method to treat a human or an animal host having ocular neovascularization induced by the expression of the normal transcription factor HIF1α gene, comprising contacting in vivo the host HIF1α genomic DNA with the chimeric oligonucleotide DNA/2′OMeRNA of claim 29.

35. An animal model comprising a mutation in the RP1 gene, mutation which has been induced by the in vivo administration of a chimeric oligonucleotide wherein said chimeric oligonucleotide is a chimeric oligonucleotide according to claim 31.

36. Use of an animal model according to claim 35 for the screening of pharmaceutical compounds capable of treating human or animal retinopathies.
